(19) United States
(12) Patent Application Publication
(10) Pub. No.: US 2025/0357097 A1
(43) Pub. Date: Nov. 20, 2025

(54) DATA DEPENDENT ACQUISITION (DDA) MASS SPECTROMETRY

(71) Applicant: Thermo Fisher Scientific (Bremen) GmbH, Bremen (DE)

(72) Inventors: Kyle Fort, Bremen (DE); Konstantin Aizikov, Bremen (DE); Tobias Woerner, Bremen (DE)

(73) Assignee: Thermo Fisher Scientific (Bremen) GmbH, Bremen (DE)

(21) Appl. No.: 19/203,737

(22) Filed: May 9, 2025

(30) Foreign Application Priority Data
May 17, 2024 (GB) 2407023.7

(51) Int. Cl. H01J 49/00; H01J 49/40; H01J 49/42

(52) U.S. Cl. CPC H01J 49/0036; H01J 49/0009; H01J 49/004; H01J 49/40; H01J 49/425

(57) ABSTRACT

Data Dependent Acquisition (DDA) mass spectrometry methods comprise ionising a sample to produce sample ions, analysing the sample ions with one or more MS1 mass analysis scan(s) to obtain MS1 data, identifying one or more precursor ions from the MS1 data, and then analysing the sample ions with one or more MS2 mass analysis scan(s). Each MS2 scan is targeted to one of the one or more precursor ions identified from the MS1 data. For each of one or more precursor ions identified from the MS1 data, a value indicative of a collision cross section (CCS) of that precursor ion is determined from the MS1 data. Based on the CCS-indicative value(s), precursor ion(s) are selected to target by the one or more MS2 mass analysis scan(s) and/or an order in which to target precursor ions by the one or more MS2 mass analysis scan(s) is determined.

### CROSS REFERENCE TO RELATED APPLICATION

This application claims priority from GB 2407023.7, filed May 17, 2024, which is incorporated herein by reference.

### FIELD

The present invention relates generally to the field of mass spectrometry, and more particularly to methods of Data Dependent Acquisition (DDA) mass spectrometry.

### BACKGROUND

The analysis and study of proteins by mass spectrometry, termed proteomics, focuses on identifying and quantifying proteins present in a certain biological state within a cell. Proteins are comprised of an amino acid chain, termed the primary sequence, and this sequence is the primary criteria that is used for protein identification in proteomic analyses. However, proteins can also be decorated by modifications such as phospho moieties or glycans, termed post-translational modifications (PTMs). These modifications have been shown to affect the resulting biology related to the modified protein, which has highlighted the need to identify these modified states and characterise the extent of modification, e.g., site-localization, modification structure, etc.

Unfortunately, these modifications occur sub-stoichiometrically, leading to a lower relative abundance as compared to their unmodified counterparts, which leads to difficulty to efficiently select and analyse these species. In order to circumvent the reduced abundance of modified proteins, both online and offline enrichment techniques have been employed whereby the protein-derived modified peptides are retained, while a majority of the unmodified peptides are discarded.

In the related field of structural biology, scientist study the higher-order structure of proteins and their complexes. There are multiple analytical tools one can use for such studies. Within the field of mass spectrometry, one such tool is that of cross-linking mass spectrometry. In cross-linking, a crosslinker, which is a small molecule with two reactive termini on opposing ends of the molecule, is introduced to the sample and these reactive ends bond with reactive amino acids along the protein backbone, covalently binding to the amino acid and crosslinker. The other reactive end of the crosslinker is able to move in the space defined by the length of the molecule. If another reactive amino-acid is present within this spatial distribution, this amino acid also binds to the molecule, linking the two locations of the protein, giving the scientist a spatial mapping of the structure of the protein/protein complex.

While providing significant structural information, this technique is also rife with analytical challenges. Namely, when the proteins are enzymatically digested to be analysed by proteomic workflows, the cross-linked peptides are present at sub-stoichiometric amounts and are therefore difficult to analyse, similar to PTMs. Analysis of cross-linked samples show the majority of the precursors selected for fragmentation are unmodified peptides rather than cross-linked peptides. Recently, the chemical nature of the crosslinker has been tailored to allow scientist to enrich for cross-linked peptides, similar to enrichment steps of phosphorylated peptides. However, this requires additional sample handling either on- or off-line from the mass spectrometer.

For both examples, an analytical challenge posed is how to identify these modified precursors and selectively target them for mass spectrometric analysis.

It is believed that there remains scope for improvements to apparatus and methods for mass spectrometry.

### SUMMARY

A first aspect provides a method of mass spectrometry comprising:
ionising a sample to produce sample ions;
(i) analysing the sample ions by performing one or more MS1 mass analysis scan(s) so as to obtain MS1 data;
(ii) identifying one or more precursor ions from the MS1 data; and
(iii) analysing the sample ions by performing one or more MS2 mass analysis scan(s), wherein each MS2 mass analysis scan is targeted to one of the one or more precursor ions identified from the MS1 data;
wherein the method further comprises:
   for each of the one or more precursor ions identified from the MS1 data: determining, from the MS1 data, a value indicative of a collision cross section (CCS) of that precursor ion; and
   selecting, based on the CCS-indicative value(s), which precursor ion(s) to target by the one or more MS2 mass analysis scan(s) and/or determining, based on the CCS-indicative value(s), an order in which to target precursor ions by the one or more MS2 mass analysis scan(s).

Embodiments provide a Data Dependent Acquisition (DDA) method of mass spectrometry in which one or more precursor ions are identified from MS1 data, and then one or more MS2 mass analysis scan(s) are triggered to target identified precursor ion(s) of interest.

In embodiments, precursor ions of interest are identified based on a value indicative of their collision cross section (CCS) (a “CCS-indicative value”) as determined from the MS1 data, and/or an order in which precursor ions are analysed by the MS2 scans is determined based on their CCS-indicative value(s). This is in contrast with conventional DDA methods, such as “Top-N” methods in which precursor ions are analysed by MS2 scans in an order that is determined based on their intensity in the MS1 data.

Embodiments build on previous work (e.g., as is described in UK Patent Application GB 2,612,580, the entire contents of which is incorporated herein by reference) in which it has been shown that CCS-indicative values can be obtained directly from MS1 data. This is in contrast with conventional methods for determining CCS values, which use dedicated ion mobility analysers, e.g. drift cells, traveling-wave ion guides, trapped ion mobility separators (TIMS), and the like, to separate ions in the CCS domain. The inventors have now recognised that the ability to determine CCS-indicative values directly from MS1 data (without ion mobility separation) enables a DDA method in which precursor ions of interest are selected and/or prioritised based on their CCS-indicative value(s). Thus, in embodiments described herein, CCS values or values indicative of CCS values as determined from the MS1 mass measurement itself are used to differentiate ions of interest and to drive an intelligent DDA workflow.

In some embodiments, for each precursor ion for which a CCS indicative value is determined from the MS1 data, this value is compared to a value or values indicative of an expected collision cross section (CCS) or an expected range of collision cross sections (CCS) for the precursor ion, and precursor ions to be targeted by the MS2 scan(s) are selected based on this comparison. Identifying precursor ions that have CCS-indicative values that deviate from an expected CCS-indicative value or range allows modified samples ions, such as in particular modified and cross-linked peptides and/or proteins, to be identified and preferentially targeted. This in turn increases the amount of instrument analysis time that is used to target such ions of interest (which can be present at low abundances and at substochiometric amounts) and reduces or minimizes the need for sample enrichment prior to MS analysis.

It will accordingly be appreciated that embodiments provide an improved method of mass spectrometry.

In the method a sample is ionised, i.e. by an ion source, to produce sample ions. The sample ions may be, e.g., peptide and/or protein ions, etc. The sample may be provided to the ion source from a separation device such as a capillary electrophoresis separation device or a chromatographic separation device (e.g. a liquid chromatography (LC) separation device or a gas chromatography (GC) separation device), etc. Additionally or alternatively, the ions may be separated by a separation device such as an ion mobility separation device, a differential ion mobility separation device, or a device configured to separate ions according to their mass to charge ratio (m/z).

The method may comprise performing a plurality of repeated cycles, wherein each cycle comprises performing the steps (i), (ii) and (iii) as described above. Thus, steps (i)-(iii) may be performed repeatedly in a cyclical manner. The method may comprise repeatedly performing cycles during a separation run of the separation device.

In a first step (i) of each cycle, the sample ions are analysed by performing one or more MS1 mass analysis scan(s) so as to obtain MS1 data, i.e. by using a mass analyser to mass analyse the sample ions. The mass analyser may be any suitable mass analyser such as an orbital trapping mass analyser, e.g. an Orbitrap™ mass analyser, a Time-of-Flight (ToF) mass analyser such as a Multi-Reflection Time-of-Flight (MR-ToF) mass analyser, an Ion Cyclotron Resonance (ICR) mass analyser, and so on.

In particular embodiments, the mass analyser is an orbital trapping mass analyser, wherein the orbital trapping mass analyser is operated with a relatively high pressure, e.g. such that the timescale for ion-gas collisions within the mass analyser is on a similar scale to the time required for a mass analysis scan. For example, an orbital trapping mass analyser is normally operated with a pressure in the region of 10⁻¹⁰ mbar or 10⁻¹¹ mbar (i.e. so as to minimise ion-gas collisions during each mass analysis scan), but in embodiments an orbital trapping mass analyser is operated with a pressure ≥10⁻⁹ mbar (e.g. between 1×10⁻⁹ and 9×10⁻⁹ mbar) when obtaining the MS1 data (such that ion-gas collisions are significant during each mass analysis scan). As is described in UK Patent Application GB 2,612,580, operating the orbital trapping mass analyser in this pressure regime allows CCS values of ions to be estimated from the width of ion peaks in the MS1 data.

In each cycle, one or more precursor ions are identified from the MS1 data, and then the sample ions are analysed by performing one or more MS2 mass analysis scan(s), with each MS2 mass analysis scan being targeted to one of the one or more precursor ions identified from the MS1 data. Precursor ions may be initially identified from the MS1 data in any suitable manner, e.g. using suitable data processing and/or peak detection.

The step of (ii) identifying one or more precursor ions in the MS1 data may comprise identifying a plurality of different precursor ions in the MS1 data. Then, the step of (iii) analysing the sample ions by performing one or more MS2 mass analysis scan(s) may comprise performing a plurality of MS2 mass analysis scans, wherein each MS2 mass analysis scan of the plurality of MS2 mass analysis scans is targeted to a different one of the plurality of different precursor ions identified in the MS1 data.

In each MS2 mass analysis scan, the method may comprise: isolating the targeted precursor ions from the sample ions; fragmenting the isolated precursor ions so as to produce fragment ions; and using a mass analyser to mass analyse the fragment ions. The step of isolating the targeted precursor ions from the sample ions may comprise using a mass filter, such as a quadrupole mass filter, to select the targeted precursor ions according to their mass to charge ratio (m/z). Sample ions having m/z within a narrow (e.g. ≤5 Da, such as ˜2D a) isolation window centred on the m/z of the targeted precursor ion may be transmitted while sample ions having m/z outside the isolation window may be attenuated. Each MS2 mass analysis scan of the plurality of MS2 mass analysis scans may be targeted to each different one of the plurality of different precursor ions by altering the centre m/z of the isolation window for each different targeted precursor ion.

In some embodiments, e.g. where the mass analyser is an orbital trapping mass analyser, the method may comprise performing a single MS1 mass analysis scan in each cycle so as to obtain the MS1 data followed by performing a single MS2 mass analysis scan in respect of each of the one or more targeted precursor ions. However, this is not necessary, and e.g. where a different type of mass analyser is used (such as, e.g., a ToF mass analyser which forms individual mass spectra by averaging the results of multiple scans) and/or where more complex methods of analysis are used, more than one MS1 mass analysis scan may be used in a cycle to obtain the MS1 data, and/or more than one MS2 mass analysis scan may be performed in respect of each of the one or more targeted precursor ions.

In the method, for each of one or more precursor ions identified from the MS1 data, a value indicative of a collision cross section (CCS) (i.e. a “CCS-indicative-value”) of that precursor ion is determined from the MS1 data. A CCS-indicative-value may be determined for some, most or all precursor ions apparent from the MS1 data.

The CCS-indicative-value may be the CCS value itself, or some other value indicative of the CCS value of a precursor ion. Each CCS-indicative-value is determined (directly) from the MS1 data, e.g. from a single MS1 spectrum (and without any reference to ion mobility drift time).

In particular embodiments, the value indicative of a collision cross section (CCS) of a precursor ion is a width of an ion peak in the MS1 data associated with that precursor ion (and the subsequent steps of using the CCS-indicative-value are performed using this width). Alternatively, the actual collision cross section (CCS) value or some other value indicative of the collision cross section (CCS) of a precursor ion may be determined from the width of an ion peak in the MS1 data associated with that precursor ion (and the subsequent steps of using the CCS-indicative-value may be performed using this value). In embodiments, any suitable measure of ion peak width may be used, such as for example the full width at half maximum (FWHM). These embodiments are particularly but not exclusively suited to embodiments where, e.g. as is described in UK Patent Application GB 2,612,580, the mass analyser is an orbital trapping mass analyser operated in a suitable pressure regime to allow CCS values of ions to be estimated from the width of ion peaks in the MS1 data.

Other methods of determining or estimating CCS-indicative values from MS1 data (without using ion mobility separation) could however be used. For example, UK Patent Application No. GB 2303690.8, the entire contents of which are incorporated herein by reference, describes a method in which CCS-indicative values are determined using a Time-of-Flight (ToF) mass analyser. This is done by obtaining two sets of data (i.e. by performing two scans (or two sets of scans)), where one or both of (i) the ion path length and (ii) the gas pressure in the ion path is changed between the two sets of data. By comparing the intensity of corresponding ion peaks in the two sets of data, the CCS of the ions giving rise to the corresponding ion peaks can be determined.

Thus, the mass analyser may be a time-of-flight (ToF) mass analyser (such as a Multi-Reflection Time-of-Flight (MR-ToF) mass analyser) that is configured to determine the mass to charge ratio (m/z) of ions by determining flight times of ions along an ion path, and the step of (i) analysing the sample ions by performing one or more MS1 mass analysis scan(s) so as to obtain MS1 data may comprise:
operating the mass analyser in a first mode of operation, and analysing sample ions by determining flight times of the sample ions along the ion path so as to obtain a first set of MS1 data, wherein in the first mode of operation (i) the ion path has a first path length, and (ii) the ion path is maintained at a first pressure; and
operating the mass analyser in a second mode of operation, and analysing sample ions by determining flight times of the sample ions along the ion path so as to obtain a second set of MS1 data, wherein in the second mode of operation (i) the ion path has a second path length, and (ii) the ion path is maintained at a second pressure, wherein the second path length is different to the first path length and/or the second pressure is different to the first pressure.

In these embodiments, the second path length may be greater than the first path length and/or the second pressure may be greater than the first pressure. In particular, the time-of-flight mass analyser may comprise one or more ion reflectors (e.g. of the various types described in GB 2303690.8), and in the first mode of operation ions may be caused to make n reflection(s) in the one or more ion reflectors, wherein n is an integer ≥0, and in the second mode of operation ions may be caused to make m reflection(s) in the one or more ion reflectors, wherein m is an integer >n.

In these embodiments, the step of determining, from the MS1 data, a value indicative of a collision cross section (CCS) of a precursor ion may comprise: comparing an intensity of a precursor ion peak in the first set of data to an intensity of the corresponding precursor ion peak in the second set of data, and determining, on the basis of the comparison, a value indicative of the CCS of the precursor ion. The method may comprise: determining a ratio of the intensity of the precursor ion peak in the first set of data to the intensity of the corresponding precursor ion peak in the second set of data, and determining the CCS-indicative value for the precursor ion using the ratio. For example, in some embodiments, the value indicative of a collision cross section (CCS) of a precursor ion is this ratio (and the subsequent steps of using the CCS-indicative-value are performed using this ratio). Alternatively, the actual collision cross section (CCS) value may be determined from the ratio, e.g. by comparing the ratio with a calibration to determine the CCS of the precursor ion (and the subsequent steps of using the CCS-indicative-value may be performed using this CCS value).

In general, in the methods described herein, the CCS-indicative values (e.g. as determined for plural precursor ions identified in the MS1 data) are used to select which precursor ion(s) of the plural precursor ions identified in the MS1 data should be targeted by the one or more MS2 mass analysis scan(s) and/or to determine an order in which the precursor ions should be targeted by the one or more MS2 mass analysis scan(s). Then, in step (iii), the one or more MS2 mass analysis scan(s) are performed, wherein each MS2 mass analysis scan is targeted to one of the selected precursor ion(s) and/or wherein the precursor ion(s) are targeted in the determined order.

The method may comprise (a) selecting which precursor ion(s) of the identified precursor ions (i.e. selecting some but not all of the identified precursor ions) to target by the MS2 mass analysis scan(s) based on the CCS-indicative value(s) (without determining an order); or (b) determining an order in which to target precursor ions by the MS2 mass analysis scan(s) based on the CCS-indicative value(s) (without selecting only some of the precursor ion(s) of the identified precursor ions to target); or (c) both selecting which precursor ion(s) of the identified precursor ions (i.e. selecting some but not all of the identified precursor ions) to target by the MS2 mass analysis scan(s) based on the CCS-indicative value(s) and determining an order in which to target the selected precursor ions by the MS2 mass analysis scan(s) based on the CCS-indicative value(s).

Other factors may be taken into account (in combination with the CCS-indicative value(s)) when selecting precursor ions and/or when determining the order.

Thus, the method may comprise selecting, based on the CCS-indicative value(s) and on one or more other factors, which precursor ion(s) to target by the one or more MS2 mass analysis scan(s) and/or determining, based on the CCS-indicative value(s) and on one or more other factors, an order in which to target precursor ions by the one or more MS2 mass analysis scan(s). The one or more other factors may include, for example, the intensity of the precursor ion in the MS1 data and/or the m/z of the precursor ion in the MS1 data, etc. The one or more other factors may also or instead include a number of time(s) that the precursor ion has already been targeted by a MS2 mass analysis scan and/or the time elapsed since the precursor ion was previously targeted by a MS2 mass analysis scan. For example, precursor ion(s) that have been targeted by a desired number of (one or more) MS2 scan(s) may be added to an exclusion list, which may be time limited. As such, it should be noted that precursor ion(s) that would be selected based (purely) on their CCS-indicative value(s) in the manner described above, below and elsewhere herein may in fact be excluded from being targeted by the MS2 mass analysis scan(s) (i.e. due to the one or more other factor(s)).

The selection of which precursor ion(s) of the identified precursor ions to target by the MS2 mass analysis scan(s) based on the CCS-indicative value(s) may be done in any suitable manner. For example, (only) those precursor ion(s) whose CCS-indicative value is greater than a threshold value and/or less than a threshold value and/or inside or outside of a range may be selected, or a certain fixed number or proportion of identified precursor ions with the highest or lowest CCS-indicative value or closest to a target CCS-indicative value may be selected.

Similarly, the determination of an order in which the precursor ions are to be targeted by the MS2 mass analysis scan(s) based on the CCS-indicative value(s) may be done in any suitable manner. For example, the precursor ions may be ordered in terms of highest to lowest CCS-indicative value, lowest to highest CCS-indicative value, etc.

In some embodiments, the selected precursor ions are precursor ions having a particular chemical class. For example, where the sample is a heterogenous mixture of different chemical classes (e.g. comprising two or more of peptides, lipids, steroids, etc.) each of the different chemical classes may show different trendlines in the CCS-indicative value-m/z space. In this regard, it has been recognised that there is a relationship between CCS-indicative value and m/z for ions of various different chemical classes. These relationships take the form of a “trend line” in respect of each different chemical class. In practice, there will be some spread of CCS-indicative values for ions having a particular chemical class and a particular m/z, but typically this spread is sufficiently small that ions of different chemical classes can be distinguished in most of the CCS-indicative value-m/z space. Thus, in embodiments, precursor ions from only one of the chemical classes may be selected for analysis and/or may be preferentially targeted by the one or more MS2 mass analysis scan(s), e.g. by selecting precursor ion(s) based on their CCS-indicative value(s) and their m/z and/or determining the order based on CCS-indicative value(s) and m/z.

In particular embodiments, the method comprises, for each of one or more of the precursor ion(s) for which a CCS indicative value is determined: comparing the determined CCS indicative value to a value or values indicative of an expected collision cross section (CCS) or an expected range of collision cross sections for that precursor ion. Such a comparison may be made for one or more or most or all precursor ions for which a CCS indicative value is determined. Then, the step of selecting which precursor ion(s) to target by the MS2 mass analysis scan(s) may comprise: selecting, based on the comparison(s), which precursor ion(s) of the identified precursor ions to target by the one or more MS2 mass analysis scan(s). Equally, the step of determining an order in which to target precursor ions by the MS2 mass analysis scan(s) may comprise: determining, based on the comparison(s), an order in which to target precursor ions by the one or more MS2 mass analysis scan(s).

In some embodiments, the method may comprise, for each of one or more or most or all of the precursor ion(s) for which a CCS indicative value is determined: calculating a difference between the determined CCS indicative value and the expected CCS indicative value. Then, (only) those precursor ion(s) for whom the calculated difference is greater than (or less than) a threshold value may be selected, or a certain fixed number or proportion of identified precursor ions with the highest or lowest calculated difference may be selected. Additionally or alternatively, the order may be determined by ordering precursor ions from highest to lowest calculated difference or from lowest to highest calculated difference.

In particular embodiments, the step of comparing may comprise, for each of one or more or most or all of the precursor ion(s) for which a CCS indicative value is determined: determining whether the determined CCS indicative value (a) is greater than a maximum expected CCS indicative value for that precursor ion. Then, the step of selecting based on the comparison may comprise: when it is determined that (a) the determined CCS indicative value is greater than the maximum expected CCS indicative value: selecting that precursor ion to be targeted by one of the MS2 mass analysis scan(s). Then, in step (iii), one of the MS2 mass analysis scan(s) may be targeted to that selected precursor ion. Additionally or alternatively, the step of determining an order based on the comparison may comprise: when it is determined that (a) the determined CCS indicative value is greater than the maximum expected CCS indicative value: giving a relatively high priority to that precursor ion in the order. The method may optionally comprise, when it is determined that (a) the determined CCS indicative value is less than the maximum expected CCS indicative value: not selecting that precursor ion (and not targeting that precursor ion by the MS2 mass analysis scan(s)) and/or giving a relatively low priority to that precursor ion in the order.

Additionally or alternatively, the step of comparing may comprise, for each of one or more or most or all of the precursor ion(s) for which a CCS indicative value is determined: determining whether the determined CCS indicative value (b) is less than a minimum expected CCS indicative value for that precursor ion. Then, the step of selecting based on the comparison may comprise: when it is determined that (b) the determined CCS indicative value is less than the minimum expected CCS indicative value: selecting that precursor ion to be targeted by one of the MS2 mass analysis scan(s). Then, in step (iii), one of the MS2 mass analysis scan(s) may be targeted to that selected precursor ion. Additionally or alternatively, the step of determining an order based on the comparison may comprise: when it is determined that (b) the determined CCS indicative value is less than the minimum expected CCS indicative value: giving a relatively high priority to that precursor ion in the order. The method may optionally comprise, when it is determined that (b) the determined CCS indicative value is greater than the minimum expected CCS indicative value: not selecting that precursor ion (and not targeting that precursor ion by the MS2 mass analysis scan(s)) and/or giving a relatively low priority to that precursor ion in the order.

Additionally or alternatively, the step of comparing may comprise, for each of one or more or most or all of the precursor ion(s) for which a CCS indicative value is determined: determining whether the determined CCS indicative value (c) falls outside an expected range of CCS indicative values for that precursor ion. Then, the step of selecting based on the comparison may comprise: when it is determined that (c) the determined CCS indicative value falls outside the expected range of CCS indicative values: selecting that precursor ion to be targeted by one of the MS2 mass analysis scan(s). Then, in step (iii), one of the MS2 mass analysis scan(s) may be targeted to that selected precursor ion. Additionally or alternatively, the step of determining an order based on the comparison may comprise: when it is determined that (c) the determined CCS indicative value falls outside the expected range of CCS indicative values: giving a relatively high priority to that precursor ion in the order. The method may optionally comprise, when it is determined that (c) the determined CCS indicative value falls inside the expected range of CCS indicative values: not selecting that precursor ion (and not targeting that precursor ion by the MS2 mass analysis scan(s)) and/or giving a relatively low priority to that precursor ion in the order.

In these embodiments, precursor ions with a relatively high priority in the order will be targeted in step (iii) by an MS2 scan before precursor ions with a relatively low priority.

As described above and elsewhere herein, identifying precursor ions that have CCS-indicative values that deviate from an expected CCS-indicative value or range in this manner allows modified samples ions, such as modified and cross-linked peptides and/or proteins, to be identified and preferentially targeted. For example, in particular embodiments, precursor ions that have CCS-indicative values that are higher than expected may be cross-linked peptides or glycosylated peptides. Precursor ions that have CCS-indicative values that are lower than expected may be phosphorylated peptides.

In embodiments, the value or values indicative of an expected collision cross section (CCS) or range of collision cross sections may be determined from a calibration such as a calibration curve. Then, the method may comprise, for each of one or more or most or all of the precursor ion(s) for which a CCS indicative value is determined: determining a value or values indicative of an expected collision cross section (CCS) or an expected range of collision cross sections for that precursor ion from the calibration and using this value or values in the comparison step.

The calibration may comprise an expected CCS-indicative value and/or an expected range of CCS-indicative values as a function of mass to charge ratio (m/z). For example, the calibration may comprise a maximum expected CCS indicative value as a function of mass to charge ratio (m/z) together with a minimum expected CCS indicative value as a function of mass to charge ratio (m/z), which together may form an expected range of CCS indicative values as a function of mass to charge ratio (m/z), i.e. an expected region in the CCS-indicative-value-m/z space. Then, a precursor ion may be selected and/or prioritised in the order when its determined CCS-indicative-value falls outside this expected region.

In embodiments, the selected precursor ions are modified precursor ions such as modified or cross-linked peptide and/or protein ions. Then, the calibration may be generated by analysing one or more samples of unmodified precursor ions, where the precursor ions are of a same type as the sample ions to be analysed. For example, where the sample ions are peptide and/or protein ions, the calibration may be generated by analysing one or more samples of unmodified peptide and/or protein ions. The calibration may be generated by measuring a value (e.g. peak width) indicative of collision cross section for numerous unmodified precursor ions with varying m/z, and building the calibration from these measured values, e.g. by averaging, etc.

As mentioned above, in each MS2 mass analysis scan, the targeted precursor ions are fragmented. In some embodiments, the fragmentation energy used and/or the fragmentation method used in each of the plural MS2 mass analysis scans is the same. Alternatively, one or both of the fragmentation energy and/or the fragmentation method may be varied between the some or all of the plural MS2 mass analysis scans, and may be selected based on the CCS-indicative value of the targeted precursor ion, e.g. so as to obtain improved MS2 data.

Thus, the method may comprise for each of one or more or most or all of the MS2 mass analysis scan(s): selecting, based on the CCS-indicative value of the precursor ion targeted by that MS2 mass analysis scan, a fragmentation energy to use when performing the MS2 mass analysis scan (and using the selected fragmentation energy when performing the MS2 mass analysis scan). Additionally or alternatively, the method may comprise, for each of one or more or most or all of the MS2 mass analysis scan(s): selecting, based on the CCS-indicative value of the precursor ion targeted by that MS2 mass analysis scan, a fragmentation method to use when performing the MS2 mass analysis scan (and using the selected fragmentation method when performing the MS2 mass analysis scan). The fragmentation method may be selected from a plurality of possible fragmentation methods, where the plurality of possible fragmentation methods can include any combination of two of more fragmentation methods, such as, e.g., collision induced dissociation (CID), electron induced dissociation (EID), photodissociation, and so on. Numerous other types of fragmentation are possible.

In these embodiments, the fragmentation energy and/or method may be selected depending on, for example, the particular chemical class as inferred from the CCS-indicative value and m/z of the targeted precursor ion, e.g. so as to obtain improved MS2 data.

Additionally or alternatively, the fragmentation energy and/or method may be selected based on the comparison (e.g. in a corresponding manner to that described above). For example, the fragmentation energy and/or method may be selected depending on whether or not (and/or by how much) the precursor ion's CCS-indicative value deviates from an expected CCS-indicative value or range. This can allow improved MS2 data to be obtained for unmodified and modified/cross-linked peptides and/or proteins, etc.

These “decision driven fragmentation” methods can be performed together with or independently of the CCS-indicative value(s) driven DDA method described above.

Thus, a second aspect provides a method of mass spectrometry comprising:
ionising a sample to produce sample ions;
(i) analysing the sample ions by performing one or more MS1 mass analysis scan(s) so as to obtain MS1 data;
(ii) identifying one or more precursor ions from the MS1 data; and
(iii) analysing the sample ions by performing one or more MS2 mass analysis scan(s), wherein each MS2 mass analysis scan is targeted to one of the one or more precursor ions identified from the MS1 data;
wherein the method further comprises for each of the one or more precursor ions identified from the MS1 data: determining, from the MS1 data, a value indicative of a collision cross section (CCS) of that precursor ion; and
for each of one or more of the MS2 mass analysis scan(s):
   selecting, based on the CCS-indicative value of the precursor ion targeted by that MS2 mass analysis scan, a fragmentation energy to use when performing the MS2 mass analysis scan; and/or
   selecting, based on the CCS-indicative value of the precursor ion targeted by that MS2 mass analysis scan, a fragmentation method to use when performing the MS2 mass analysis scan.

This aspect can, and in embodiments does, include any one or more or each of the optional features described herein.

A further aspect provides a non-transitory computer readable storage medium storing computer software code which when executed on a processor performs the method(s) described above.

A further aspect provides a control system for an analytical instrument such as a mass spectrometer, the control system configured to cause the analytical instrument to perform the method(s) described above.

A further aspect provides an analytical instrument, such as a mass spectrometer, comprising the control system described above.

A further aspect provides an analytical instrument, such as a mass spectrometer, comprising:
an ion source configured to ionise a sample to produce sample ions;
a mass filter configured to filter ions using an isolation window;
a fragmentation device configured to fragment sample ions so as to produce fragment ions;
a mass analyser; and
a control system configured to:
(i) cause the instrument to perform one or more MS1 mass analysis scan(s) so as to obtain MS1 data;
(ii) identify one or more precursor ions from the MS1 data; and
(iii) cause the instrument to perform one or more MS2 mass analysis scan(s), wherein each MS2 scan is targeted to one of the one or more precursor ions identified from the MS1 data;
wherein the control system is further configured to:
   for each of the one or more precursor ions identified from the MS1 data: determine, from the MS1 data, a value indicative of a collision cross section (CCS) of that precursor ion; and
   select, based on the CCS-indicative value(s), which precursor ion(s) to target by the one or more MS2 mass analysis scan(s) and/or determine, based on the CCS-indicative value(s), an order in which to target precursor ions by the one or more MS2 mass analysis scan(s).

A further aspect provides an analytical instrument, such as a mass spectrometer, comprising:
an ion source configured to ionise a sample to produce sample ions;
a mass filter configured to filter ions using an isolation window;
at least one fragmentation device configured to fragment sample ions so as to produce fragment ions;
a mass analyser; and
a control system configured to:
(i) cause the instrument to perform one or more MS1 mass analysis scan(s) so as to obtain MS1 data;
(ii) identify one or more precursor ions from the MS1 data; and
(iii) cause the instrument to perform one or more MS2 mass analysis scan(s), wherein each MS2 scan is targeted to one of the one or more precursor ions identified from the MS1 data;
wherein the control system is further configured to:
   for each of the one or more precursor ions identified from the MS1 data: determine, from the MS1 data, a value indicative of a collision cross section (CCS) of that precursor ion; and
for each of one or more of the MS2 mass analysis scan(s):
   select, based on the CCS-indicative value of the precursor ion targeted by that MS2 mass analysis scan, a fragmentation energy to use when performing the MS2 mass analysis scan; and/or
   select, based on the CCS-indicative value of the precursor ion targeted by that MS2 mass analysis scan, a fragmentation method to use when performing the MS2 mass analysis scan.

These aspects can, and in some embodiments do, include any one or more or each of the optional features described herein.

### BRIEF DESCRIPTION OF THE DRAWINGS

Various embodiments will now be described in more detail with reference to the accompanying Figures, in which:

FIG. 1 shows schematically a mass spectrometer in accordance with embodiments;

FIG. 2 shows simulated data of the observed mass resolution for precursor ions measured during a proteomics analysis using an Orbitrap™ mass analyser operated using a standard ultra-high vacuum (UHV);

FIG. 3 shows simulated data of the observed mass resolution for unmodified and modified precursor ions measured during a proteomics analysis using an Orbitrap™ mass analyser operated using a standard ultra-high vacuum (UHV);

FIG. 4 shows simulated data of the observed mass resolution for ions using a standard pressure (labelled “Precursors, Std. Pressure”), together with real data measured using the CCS workflow of embodiments (labelled “Precursors, Elevated Pressure”), wherein it can be seen that when the UHV is elevated, the various charge states present in the precursors resolve into charge state-dependent trendlines due to the inherent higher CCS value of higher charge states;

FIG. 5 shows data for the 2+ charge state ions from FIG. 4, together with simulated data of the observed mass resolution for precursors that have modifications that result in the CCS value being reduced (labelled “Mod. CCS Reduced”) and for precursors that have modifications that result in the CCS value being increased (labelled “Mod., CCS Increased”);

FIG. 6 shows a simulated MS1 mass spectrum that contains 12 precursors of various intensity and m/z values (ion isotopes are not shown);

FIG. 7 shows the observed mass resolution for each of the precursors of FIG. 6 as a function of m/z, together an “Expected Resolution” corresponding to the 2+ trendline of an unmodified standard and an “Unmodified Region” which is representative of the spread observed in the mass resolution values of the 2+ ions of the unmodified standard;

FIGS. 8A-8B illustrate two selection strategies based on the data shown in FIG. 7, in which either ions that fall below the “Unmodified Region” are targeted for MS2 analysis (FIG. 8B), or ions that fall above the “Unmodified Region” are targeted for MS2 analysis (FIG. 8A); and

FIG. 9 shows schematically a DDA method of mass spectrometry in accordance with embodiments.

### DETAILED DESCRIPTION

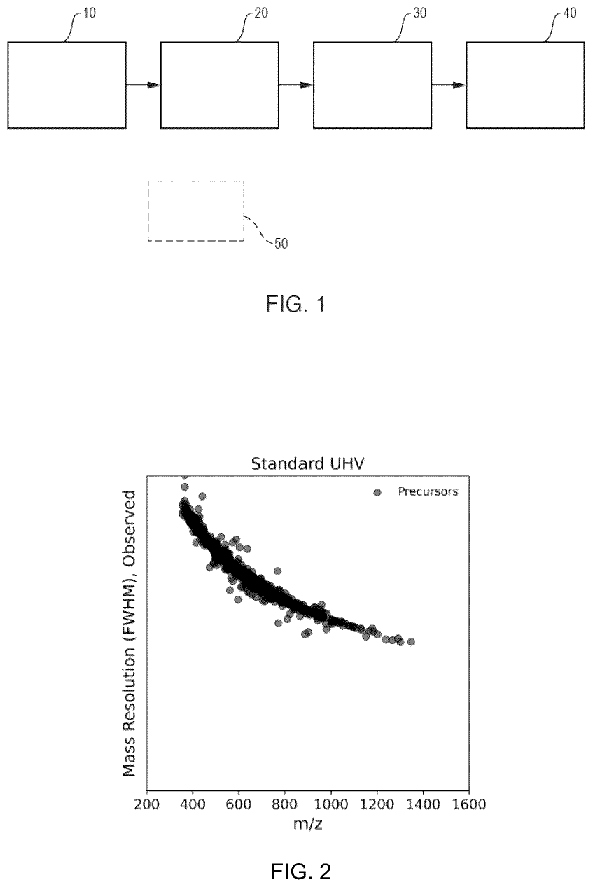

FIG. 1 illustrates schematically an analytical instrument, such as a mass spectrometer, that may be used in conjunction with the methods described herein. As shown in FIG. 1, the instrument includes an ion source 10, a mass filter 20, a fragmentation device 30, and a mass analyser 40.

The ion source 10 is configured to generate ions from a sample. The ion source 10 may be coupled to a separation device (not shown) such as a liquid chromatography (LC) separation device, a gas chromatography (GC) separation device, or a capillary electrophoresis separation device, and the like, such that the sample which is ionised in the ion source 10 comes from the separation device. The ion source 10 can be any suitable ion source, such as an electrospray ionisation (ESI) ion source, an atmospheric pressure ionisation (API) ion source, a chemical ionisation ion source, an electron impact (EI) ion source, or similar.

The analytical instrument may additionally or alternatively include an ion separation device arranged downstream of the ion source and configured to separate samples ions according to a physico-chemical property. For example, the instrument may include an ion mobility (IM) separator, a differential ion mobility separator, or a device configured to separate ions according to their mass to charge ratio (m/z)).

The mass filter 20 is arranged downstream of the ion source 10 and is configured to receive ions from the ion source 10 (optionally via the ion separation device). The mass filter 20 is configured to filter the received ions according to their mass to charge ratio (m/z). The mass filter 20 may be configured such that received ions having m/z within an m/z transmission window (or “isolation window”) of the mass filter are onwardly transmitted by the mass filter, while received ions having m/z outside the m/z transmission window are attenuated by the mass filter, i.e. are not onwardly transmitted by the mass filter. The width and/or the centre m/z of the transmission window may be controllable (variable), e.g. by suitable control of RF and/or DC voltage(s) applied to electrodes of the mass filter 20. Thus, for example, the mass filter 20 may be operable in a transmission mode of operation, whereby most or all ions within a relatively wide m/z window are onwardly transmitted by the mass filter 20, and a filtering mode of operation, whereby only ions within a relatively narrow m/z window (centred at a desired m/z) are onwardly transmitted by the mass filter 20. The mass filter 20 can be any suitable type of mass filter, such as a quadrupole mass filter.

The fragmentation device 30 is arranged downstream of the mass filter 20 and is configured to receive most or all ions transmitted by the mass filter 20. The fragmentation device 30 may be configured to selectively fragment some or all of the received ions, i.e. so as to produce fragment ions. The fragmentation device 30 may be operable in a fragmentation mode of operation, whereby most or all received ions are fragmented so as to produce fragment ions (which may then be onwardly transmitted from the fragmentation device 30), and a non-fragmentation mode of operation, whereby most or all received ions are onwardly transmitted without being (deliberately) fragmented. It would also be possible for a non-fragmentation mode of operation to be implemented by causing ions to bypass the fragmentation device 30. The fragmentation device 30 may also be operable in one or more intermediate modes of operation, e.g. whereby the degree of fragmentation is controllable (variable). The fragmentation device 30 can also be operable in higher order (MS^{N}) fragmentation modes of operation, e.g. whereby fragment ions are further fragmented one or more times by the fragmentation device 30.

The fragmentation device 30 can be any suitable type of fragmentation device, such as for example a collision induced dissociation (CID) fragmentation device, an electron induced dissociation (EID) fragmentation device, a photodissociation fragmentation device, and so on. Numerous other types of fragmentation are possible.

In some embodiments, the fragmentation device 30 is capable of performing more than one type of fragmentation method, or else more than one type of fragmentation device is provided so that the instrument can select between performing different fragmentation methods. Similarly, the fragmentation device(s) 30 may be capable of fragmenting ions using different fragmentation energies.

In some embodiments, the fragmentation device 30 is a collision induced dissociation (CID) fragmentation device. The CID fragmentation device may include a collision cell which may be filled with a collision gas, e.g. maintained at a relatively high pressure. Ions may be selectively fragmented in the collision cell by controlling (varying) the kinetic energy with which ions are caused to enter the collision cell. In a fragmentation mode of operation, ions may be accelerated so that they enter the collision cell with a relatively high kinetic energy, which may cause most or all of the accelerated ions to fragment. In a non-fragmentation mode of operation, ions may be caused to enter the collision cell with a relatively low kinetic energy, which may be insufficient to cause most or all of the ions to fragment. In intermediate modes, ions may be caused to enter the collision cell with intermediate kinetic energies.

The mass analyser 40 is arranged downstream of the fragmentation device 30 and is configured to receive ions from the fragmentation device 30. Thus, the mass analyser 40 may receive unfragmented precursor ions and/or fragment ions, depending on the mode of operation of the fragmentation device 30. The mass analyser 40 is configured to analyse the received ions so as to determine their mass to charge ratio (m/z) and/or mass, i.e. to produce a mass spectrum of the ions. The mass analyser 40 can be any suitable type of mass analyser, such as an ion trap mass analyser, an electrostatic orbital trap mass analyser (such as an Orbitrap™ FT mass analyser as made by Thermo Fisher Scientific) or a time-of-flight (ToF) mass analyser such as a multi-reflecting time-of-flight (MR-ToF) mass analyser.

It should be noted that FIG. 1 is merely schematic, and that the instrument can, and in embodiments does, include any number of one or more additional components. For example, the instrument may include one or more ion transfer stage(s) arranged between any of the illustrated components, e.g. including an atmospheric pressure interface and/or one or more ion guides, lenses and/or other ion optical devices configured such that some or all of the ions can be transmitted appropriately through the instrument. The ion transfer stage(s) may include any suitable number and configuration of ion optical devices, for example optionally including one or more ion guides, lenses and/or other ion optical devices.

In some embodiments, the instrument may include more than one mass analyser. For example, the instrument may be a dual mass analyser hybrid mass spectrometer of the type described in EP 3,410,463, the contents of which are incorporated herein by reference.

As also shown in FIG. 1, the instrument is under the control of a control unit 50, such as an appropriately programmed computer, which controls the operation of various components of the instrument and, for example, sets the voltages to be applied to the various components of the instrument. The control unit 50 may also receive and process data from various components including the analyser(s) in the manner of various embodiments.

The instrument may be operable in various mode of operation. In particular, the instrument may be a tandem mass spectrometer operable in an MS1 mode of operation and an MS2 mode of operation.

In the MS1 (or “full mass scan”) mode of operation, the mass filter 20 is operated in its transmission mode of operation and the fragmentation device 30 is operated in its non-fragmentation mode of operation, e.g. so that a wide m/z range (e.g. full mass range) of unfragmented (“precursor” or “parent”) ions are analysed by the analyser 40 to produce an MS1 spectrum.

In the MS2 mode of operation, the mass filter 20 is operated in its filtering mode of operation and the fragmentation device 30 is operated in its fragmentation mode of operation, e.g. so that a selected narrow m/z range of precursor ions are fragmented and the resulting fragment (“product” or “daughter”) ions are analysed by the analyser 40 to produce an MS2 spectrum.

The instrument may also be operable in one or more higher order fragmentation modes of operation, such as for example an MS3 mode of operation, whereby precursor ions are fragmented, at least some of the resulting fragment ions are themselves fragmented, and the second-generation fragment ions (“granddaughter ions”) are analysed by the analyser 40 produce an MS3 spectrum. In general, the instrument may be operable in any order of fragmentation mode of operation, i.e. in an MS^{N} mode of operation where N≥2.

As described above, an analytical challenge in the field of proteomics is how to identify and select modified peptide ions, such as PTMs and cross-linked peptides, in a proteomic workflow while minimizing additional sample handling requirements. A solution according to embodiments leverages the peptide-level structural changes that result from protein modification. It has been demonstrated in the literature that modifications to a peptide ion can impact its structure in the gas-phase, either by compaction, i.e. a reduction in structural area/volume as compared to an unmodified peptide, or by elongation, i.e. an increase in structural area/volume as compared to an unmodified peptide ion. These structural differences can be resolved with structurally sensitive techniques. One such technique that this widely used is ion mobility, where ions are separated with respect to their collision cross section (CCS) values, which are tied to the rotationally averaged structure the ion adopts in the gas phase.

Recently, the inventors have published work, e.g. as is described in UK Patent Application GB 2,612,580, whereby these CCS values are measured based on the mass measurement step in an Orbitrap™ mass analyser. In this work, it was shown that at elevated UHV pressures (e.g. around 10⁻⁹ mbar) in the Orbitrap™ mass analyser, the ion's decay in the transient domain is tied to its CCS value. For ensemble measurements, the decay rate of an individual ion species, i.e. an ion of a certain m/z and charge state, results in a change in the observed mass resolution (i.e. peak width) of that ion in the mass spectrum. Here, this work is extended to develop a real-time, data-driven targeted workflow where precursors that show deviations from expected mass resolution indicate that they are modified ions. These ions are then selectively targeted for MS2 analysis on the fly, increasing the utilization of instrument duty cycle for modified peptide analysis and reducing or minimizing the need for pre-analysis sample enrichment.

FIG. 2 shows simulated data of the observed mass resolution for precursor ions measured during a proteomics analysis using an Orbitrap™ mass analyser operated using a standard ultra-high vacuum (UHV). As shown in FIG. 2, the mass resolution of an ion scales with the inverse square root of the m/z value. The UHV used the mass analyser is maintained at a value (e.g. around 10⁻¹⁰ mbar or 10⁻¹¹ mbar) where the mean free path of a peptide ion is such that the ion population will not appreciably decay over the measurement and therefore have minimal loses due to collisions with the background gas. Because of this, ions with different structures and inherently different CCS values show similar observed mass resolution values as a function of m/z.

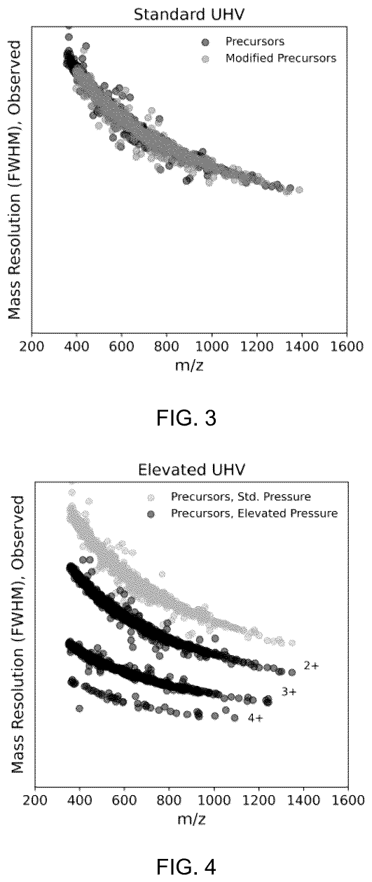

FIG. 3 shows corresponding simulated data for precursors that have structurally-influencing modifications, such as glycans, cross-links, or phospho groups, superimposed over the data from FIG. 2. Here, due to the lack of ion decay over the timeframe of the measurement, structural differences do not result in separation in the mass resolution domain. Thus, as shown by FIG. 3, even though the two ion groups are structurally unique, unmodified precursors and modified precursors overlay in mass resolution versus m/z due to the lack of collisions during the Orbitrap™ mass analyser measurement.

In order to measure CCS values in the Orbitrap™ mass analyser, the UHV is increased to where the ions start to show appreciable decay on the timescale of the transient acquisition (e.g. around 10⁻⁹ mbar). As shown in FIG. 4, the result of this decay is demonstrated in the observed mass resolution.

As can be seen from FIG. 4, the individual charge states start to group in mass resolution-m/z space, as higher charge state ions generally adopt a large CCS value. This results in higher charge state ions decaying faster, resulting in a lower observed mass resolution and separation of the various charge states in this dimension. By controlling the UHV pressure and identifying unmodified precursor ions for a known standard (e.g. Hela), these charge state-dependent trendlines can be mapped and calibrated for later use in a targeted workflow analysing an unknown compound.

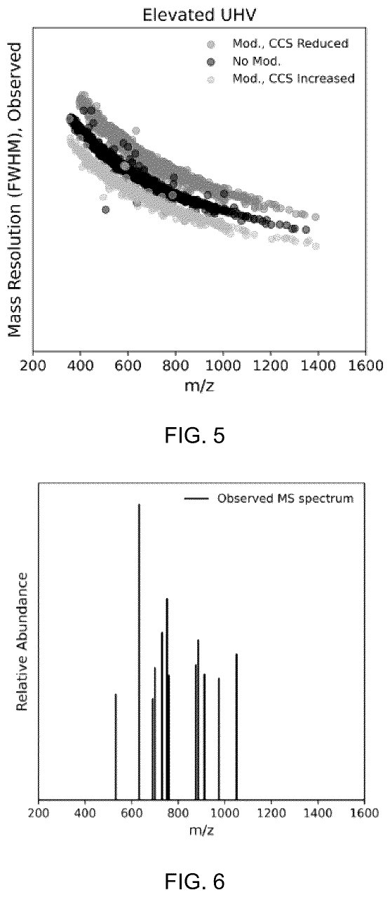

Furthermore, if one focuses only on a single charge state of FIG. 4, such as for example the 2+ charge state as shown in FIG. 5, one can show that ions that are modified can be resolved from their unmodified counterparts, either with a higher-CCS value and lower observed mass resolution (i.e. ions falling below the “No Mod” grouping in FIG. 5), or with a lower-CCS value and higher observed mass resolution (i.e. ions falling above the “No Mod” grouping in FIG. 5). In embodiments, this separation is utilised for a targeted, real-time workflow.

As discussed above, protein modifications occur at sub-stochiometric levels and would therefore result a small portion of the instrument's analysis time being dedicated to their analysis with conventional stochastic acquisition. FIG. 6 shows an example simulated MS1 mass spectrum, e.g. as would be acquired during a proteomic workflow. In this example, the MS1 scan shows 12 precursor signals with various m/z and intensity values. In standard proteomic workflows, such as DDA, the “Top N” most intense of these precursors (isotopes not shown) would be selected for fragmentation, ignoring their modification status.

However, in accordance with embodiments, the elevated UHV pressure can be utilised to map the observed mass resolutions in real time.

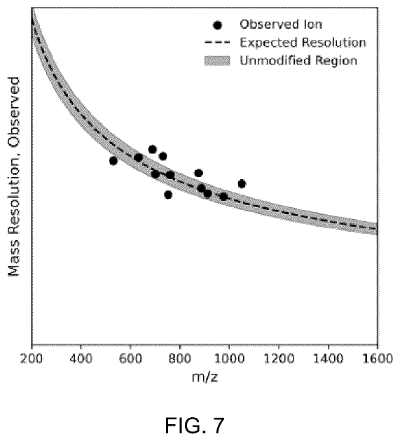

FIG. 7 shows the observed mass resolution for each of the precursors of FIG. 6 as a function of m/z. Additionally, the 2+ trendline of the unmodified standard presented in FIG. 5 is shown by the “Expected Resolution” and the “Unmodified Region”, which is representative of the spread observed in the mass resolution values of the 2+ ions of the unmodified standard.

Utilizing the previous established “Expected Resolution” line for unmodified peptide ions (from a known compound such as Hela), one can see that 6 precursor ions demonstrate a mass resolution outside of the expected “Unmodified Region”. This data suggests that these 6 precursors are structurally unique, and this could be due to the presence of modifications such as phosphorylation or cross-linking.

Building this knowledge into a data-driven workflow, this allows a user to selectively target higher CCS value precursors, e.g. for cross-linking analysis or glycan analysis, or to target lower CCS value precursors for modifications that lead to compaction, e.g. phosphorylation. In such a workflow, the precursors targeted will be the precursors that fall outside of the “Unmodified Region” and therefore a significantly higher percentage of the instrument's analysis time will be directed to precursors that are likely to exhibit the modification of interest.

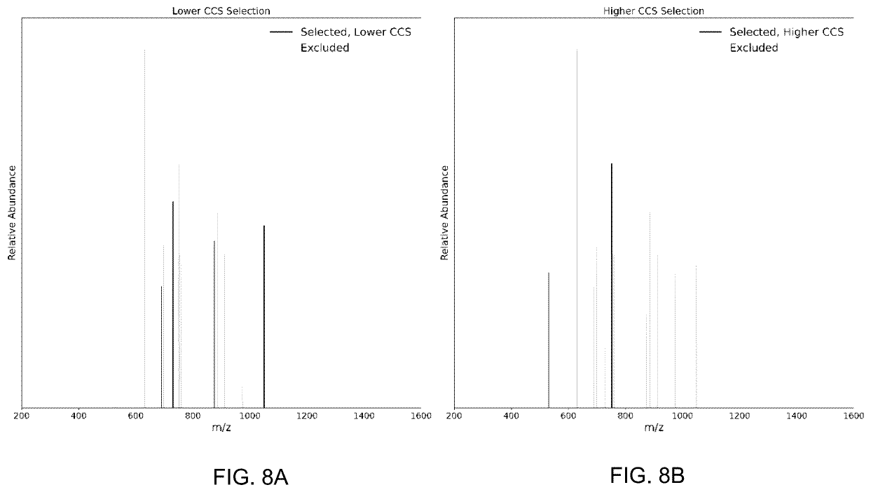

FIGS. 8A-8B illustrate two selection strategies based on the data shown in FIG. 7. If the user is interested in studying modifications that are known to increase the CCS value of the ion, ions that fall below the “Unmodified Region” can be targeted (FIG. 8B), while all other ions may be excluded. Conversely, if the user is interested in modifications that traditionally exhibit CCS compaction, such as phosphorylation, the method can target values that fall above the “Unmodified Region” for subsequent MS2 analysis (FIG. 8A).

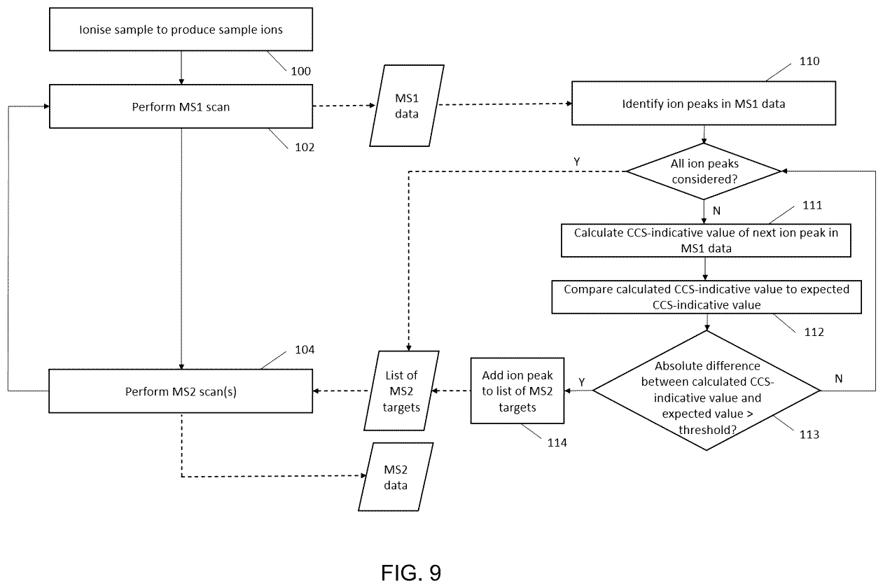

FIG. 9 illustrates a method of operating the analytical instrument of FIG. 1 in accordance with embodiments. A sample is provided to a (e.g. LC) separation device so that the sample is separated, and the eluent from the separation device is ionised (step 100) in the ion source 10. The resulting ions are analysed by the instrument operating in a data dependent acquisition (DDA) mode of operation. Additionally or alternatively, sample ions may be separated by a separation device such as an ion mobility separation device.

The data dependent acquisition (DDA) mode of operation involves the instrument repeatedly performing, during a separation run of the separation device, the steps of: (i) obtaining an MS1 spectrum across an m/z range of interest (step 102); (ii) identifying one or more precursor ions of interest in the MS1 spectrum; and (iii) obtaining an MS2 (or MS^{N}) spectrum in respect of each the identified precursor ions of interest (step 104).

Step (i) comprises the instrument performing one or more MS1 mass analysis scans to generate the MS1 spectrum. Step (iii) comprises, for each of the identified precursor ions of interest: isolating the precursor ion using the mass filter 20, fragmenting the isolated precursor ions in the fragmentation device 30, and mass analysing the fragment ions using the mass analyser 40. Thus, during each cycle, multiple MS2 spectra (or, more generally, multiple MS^{N} spectra) are acquired by sequentially altering the centre of the mass filter's (narrow) m/z window between each of a plurality of different target m/z values, e.g. so as to sequentially select (and fragment) each of a plurality of different precursor ions with respective different m/z. The plurality of different m/z values correspond to the plurality of different targeted precursor ions.

As shown in FIG. 9, in accordance with embodiments, a list of target precursor ions is generated based on CCS-indicative values determined for each ion peak in the MS1 data. Thus, the instrument identifies peaks in the MS1 spectrum (step 110) and calculate the CCS-indicative value of each identified peak in the MS1 spectrum (step 111). Then, the calculated CCS-indicative value for each peak is compared to an expected CCS-indicative value or expected range of CCS-indicative values for that peak (step 112). As described above, the expected CCS-indicative value can be determined from a calibration curve (e.g. as shown in FIG. 7), which may have been created by analysing one or more samples of unmodified precursor ions.

In step 113, where the peak has a CCS-indicative value sufficiently similar to the expected value, that peak may be ignored or assigned a relatively low priority. Where the peak has a CCS-indicative value sufficiently different from the expected value, that peak may be added to a list of targets (in step 114) and/or assigned a relatively high priority. Once all peaks in the MS1 spectrum have been considered, a complete list of MS2 targets, optionally together with a priority order, may be assembled and used to direct the MS2 scans in step 104. This process may then be repeated for the next MS1 spectrum, and so on.

It should be noted that FIG. 9 shows a simplified method, and that in practice additional steps may be provided in the workflow. In particular, additional logic steps may be provided between step 102 and step 104, e.g. in the manner of standard DDA workflows, such as for example a charge state determination/inclusion check, an intensity ranking (“Top-N”) step, and/or the use of an exclusion list or lists to exclude MS2 targets that have already or recently been analysed.

It will accordingly be understood that, in embodiments, differences in CCS-driven observed mass resolutions are used to target precursors that fall above or below an expected unmodified region. The CCS-driven decision making regarding which ions to target can increase the amount of instrument analysis time that is used to target ions of interest, which can be present at low abundances and at substochiometric amounts. This can beneficially reduce or minimize the need to do sample enrichment prior to MS analysis.

Although particular embodiments have been described in detail above, various alternatives are possible. For example, CCS-indicative values could instead be determined from the MS1 data using the method described in UK Patent Application No. GB 2303690.8. In this method, CCS-indicative values are determined using a Time-of-Flight (ToF) mass analyser, where two sets of data are obtained (i.e. by performing two scans (or two sets of scans)), where one or both of (i) the ion path length and (ii) the gas pressure in the ion path is changed between the two sets of data. By comparing the intensity of corresponding ion peaks in the two sets of data, the CCS of the ions giving rise to the corresponding ion peaks can be determined.

In some embodiments, the selected precursor ions can be precursor ions having a particular chemical class. For example, where the sample is a heterogenous mixture of different chemical classes (e.g. comprising two or more of peptides, lipids, steroids, etc.) each of the different chemical classes may show different trendlines in the CCS-indicative value-m/z space. Then, precursor ions from only one of the chemical classes may be selected for analysis and/or may be preferentially targeted by the one or more MS2 mass analysis scan(s) by selecting precursor ion(s) based on their CCS-indicative value(s) and their m/z and/or determining the order based on CCS-indicative value(s) and m/z.

The methods described herein can also be utilised to trigger different MS2 fragmentation energies or methods. That is, different fragmentation strategies can be chosen depending on the CCS-indicative value. For example, referring again to FIG. 7, if there are both peptides that fall in the unmodified region and peptides that have a lower CCS-indicative values and that therefore may be phosphorylated, the phosphorylated peptides may be selected and fragmented with one fragmentation method and/or energy (e.g. EID at a certain electron energy) and the unmodified peptides may be later selected and fragmented with a different fragmentation method and/or energy (e.g. HCD or EID at a different electron energy) as compared to the phosphorylated case. Similarly, for the case of a heterogenous mixture of different chemical classes (e.g. peptide, lipid, steroid as mentioned above), different fragmentation methods and/or energies may be used for different chemical classed, e.g. one fragmentation strategy may be used for peptides and another for steroids, etc.

Although the present invention has been described with reference to various embodiments, it will be understood that various changes may be made without departing from the scope of the invention as set out in the accompanying claims.

We claim:

1. A method of mass spectrometry comprising:
ionising a sample to produce sample ions;
(i) analysing the sample ions by performing one or more MS1 mass analysis scan(s) so as to obtain MS1 data;
(ii) identifying one or more precursor ions from the MS1 data; and
(iii) analysing the sample ions by performing one or more MS2 mass analysis scan(s), wherein each MS2 mass analysis scan is targeted to one of the one or more precursor ions identified from the MS1 data;
wherein the method further comprises:
for each of one or more precursor ions identified from the MS1 data:
determining, from the MS1 data, a value indicative of a collision cross section (CCS) of that precursor ion; and
selecting, based on the CCS-indicative value(s), which precursor ion(s) to target by the one or more MS2 mass analysis scan(s) and/or determining, based on the CCS-indicative value(s), an order in which to target precursor ions by the one or more MS2 mass analysis scan(s).

2. The method of claim 1, wherein the method comprises:
for each of one or more of the precursor ion(s) for which a CCS-indicative value is determined: comparing the determined CCS-indicative value to a value or values indicative of an expected collision cross section (CCS) or range of collision cross sections for that precursor ion; and
selecting, based on the comparison(s), which precursor ion(s) to target by the one or more MS2 mass analysis scan(s) and/or determining, based on the comparison(s), the order in which to target precursor ions by the one or more MS2 mass analysis scan(s).

3. The method of claim 2, wherein:
the step of comparing comprises, for each of one or more of the precursor ion(s) for which a CCS-indicative value is determined: determining whether the determined CCS-indicative value (a) is greater than a maximum expected CCS-indicative value for that precursor ion; and/or (b) is less than a minimum expected CCS-indicative value for that precursor ion; and/or (c) falls outside an expected range of CCS-indicative values for that precursor ion; and
the step of selecting based on the comparison(s) comprises: when it is determined that (a) the determined CCS-indicative value is greater than the maximum expected CCS-indicative value; and/or (b) the determined CCS-indicative value is less than the minimum expected CCS-indicative value; and/or (c) the determined CCS-indicative value falls outside the expected range of CCS-indicative values: selecting that precursor ion; and/or
the step of determining the order based on the comparison(s) comprises: when it is determined that (a) the determined CCS-indicative value is greater than the maximum expected CCS-indicative value; and/or (b) the determined CCS-indicative value is less than the minimum expected CCS-indicative value; and/or (c) the determined CCS-indicative value falls outside the expected range of CCS-indicative values: giving a relatively high priority to that precursor ion in the order.

4. The method of claim 3, wherein:
the step of selecting based on the comparison(s) comprises: when it is determined that (a) the determined CCS-indicative value is less than the maximum expected CCS-indicative value; and/or (b) the determined CCS-indicative value is greater than the minimum expected CCS-indicative value; and/or (c) the determined CCS-indicative value falls inside the expected range of CCS-indicative values: not selecting that precursor ion; and/or
the step of determining the order based on the comparison(s) comprises: when it is determined that (a) the determined CCS-indicative value is less than the maximum expected CCS-indicative value; and/or (b) the determined CCS-indicative value is greater than the minimum expected CCS-indicative value; and/or (c) the determined CCS-indicative value falls inside the expected range of CCS-indicative values: giving a relatively low priority to that precursor ion in the order.

5. The method of claim 1, wherein the precursor ion(s) that are selected and/or that are given a relatively high priority in the order are modified precursor ions.

6. The method of claim 5, wherein the sample ions are peptide and/or protein ions, and wherein the precursor ion(s) that are selected and/or that are given a relatively high priority in the order are modified or cross-linked peptide and/or protein ions.

7. The method of claim 2, further comprising determining the value or values indicative of an expected collision cross section (CCS) or range of collision cross sections from a calibration, wherein the calibration is a calibration generated by analysing one or more samples of unmodified precursor ions.

8. The method of claim 1, further comprising, for each of one or more of the MS2 mass analysis scan(s):
selecting, based on the CCS-indicative value of the precursor ion targeted by that MS2 mass analysis scan, a fragmentation energy to use when performing the MS2 mass analysis scan; and/or
selecting, based on the CCS-indicative value of the precursor ion targeted by that MS2 mass analysis scan, a fragmentation method to use when performing the MS2 mass analysis scan.

9. The method of claim 1, wherein:
the value indicative of a collision cross section (CCS) of a precursor ion is a width of an ion peak in the MS1 data that corresponds to the precursor ion; and/or
the step of determining a value indicative of a collision cross section (CCS) of a precursor ion comprises: determining a value indicative of the collision cross section (CCS) of the precursor ion from a width of an ion peak in the MS1 data that corresponds to the precursor ion.

10. The method of claim 1, wherein the step of (i) analysing the sample ions by performing one or more MS1 mass analysis scan(s) comprises using an orbital trapping mass analyser to mass analyse the sample ions, wherein the orbital trapping mass analyser is operated with a pressure ≥10⁻⁹ mbar.

11. The method of claim 1, wherein:
the step of (i) analysing the sample ions by performing one or more MS1 mass analysis scan(s) comprises: using a time-of-flight (ToF) mass analyser to mass analyse the sample ions, wherein the time-of-flight (ToF) mass analyser is configured to determine the mass to charge ratio (m/z) of ions by determining flight times of ions along an ion path;
the step of using a time-of-flight (ToF) mass analyser to mass analyse the sample ions comprises:
operating the mass analyser in a first mode of operation, and analysing sample ions by determining flight times of the sample ions along the ion path so as to obtain a first set of MS1 data, wherein in the first mode of operation (i) the ion path has a first path length, and (ii) the ion path is maintained at a first pressure; and
operating the mass analyser in a second mode of operation, and analysing sample ions by determining flight times of the sample ions along the ion path so as to obtain a second set of MS1 data, wherein in the second mode of operation (i) the ion path has a second path length, and (ii) the ion path is maintained at a second pressure, wherein the second path length is different to the first path length and/or the second pressure is different to the first pressure; and
the step of determining, from the MS1 data, a value indicative of a collision cross section (CCS) of a precursor ion comprises:
comparing an intensity of a precursor ion peak in the first set of data to an intensity of a corresponding precursor ion peak in the second set of data; and
determining, on the basis of the comparison, a value indicative of the CCS of the precursor ion.

12. The method of claim 1, wherein:
the step of (ii) identifying one or more precursor ions from the MS1 data comprises identifying a plurality of different precursor ions from the MS1 data; and
the step of (iii) analysing the sample ions by performing one or more MS2 mass analysis scan(s) comprises performing a plurality of MS2 mass analysis scans, wherein each MS2 mass analysis scan of the plurality of MS2 mass analysis scans is targeted to a different one of the plurality of different precursor ions identified from the MS1 data.

13. The method of claim 1, wherein the method comprises, for each MS2 mass analysis scan:
isolating the targeted precursor ions from the sample ions;
fragmenting the isolated precursor ions of interest so as to produce fragment ions; and
mass analysing the fragment ions so as to obtain MS2 data.

14. The method of claim 13, wherein the step of isolating the targeted precursor ions from the sample ions comprises using a mass filter to select the targeted precursor according to their mass to charge ratio (m/z).

15. The method of claim 1, wherein the method comprises performing a plurality of repeated cycles, wherein each cycle comprises performing steps (i), (ii) and (iii).

16. The method of claim 15, wherein:
the sample is provided from a separation device and/or the sample ions are separated by a separation device; and
the method comprises repeatedly performing cycles during a separation run of the separation device.

17. A non-transitory computer readable storage medium storing computer software code which when executed on a processor performs the method of claim 1.

18. A control system for an analytical instrument, the control system configured to cause the analytical instrument to perform the method of claim 1.

19. An analytical instrument comprising:
an ion source configured to ionise a sample to produce sample ions;
a mass filter configured to filter ions using an isolation window;
a fragmentation device configured to fragment sample ions so as to produce fragment ions;
a mass analyser; and
a control system configured to:
(i) cause the instrument to perform one or more MS1 mass analysis scan(s) so as to obtain MS1 data;
(ii) identify one or more precursor ions from the MS1 data; and
(iii) cause the instrument to perform one or more MS2 mass analysis scan(s), wherein each MS2 scan is targeted to one of the one or more precursor ions identified from the MS1 data;
wherein the control system is further configured to:
for each of the one or more precursor ions identified from the MS1 data:
determine, from the MS1 data, a value indicative of a collision cross section (CCS) of that precursor ion; and
select, based on the CCS-indicative value(s), which precursor ion(s) to target by the one or more MS2 mass analysis scan(s) and/or determine, based on the CCS-indicative value(s), an order in which to target precursor ions by the one or more MS2 mass analysis scan(s).

20. An analytical instrument comprising:
an ion source configured to ionise a sample to produce sample ions;
a mass filter configured to filter ions using an isolation window;
at least one fragmentation device configured to fragment sample ions so as to produce fragment ions;
a mass analyser; and
a control system configured to:
(i) cause the instrument to perform one or more MS1 mass analysis scan(s) so as to obtain MS1 data;
(ii) identify one or more precursor ions from the MS1 data; and
(iii) cause the instrument to perform one or more MS2 mass analysis scan(s), wherein each MS2 scan is targeted to one of the one or more precursor ions identified from the MS1 data;
wherein the control system is further configured to:
for each of one or more precursor ions identified from the MS1 data:
determine, from the MS1 data, a value indicative of a collision cross section (CCS) of that precursor ion; and
for each of one or more of the MS2 mass analysis scan(s):
select, based on the CCS-indicative value of the precursor ion targeted by that MS2 mass analysis scan, a fragmentation energy to use when performing the MS2 mass analysis scan; and/or
select, based on the CCS-indicative value of the precursor ion targeted by that MS2 mass analysis scan, a fragmentation method to use when performing the MS2 mass analysis scan.
